(19) United States
(12) Patent Application Publication
(10) Pub. No.: US 2014/0172003 A1
(43) Pub. Date: Jun. 19, 2014

(54) THREADED BALLOON CATHETER SEAL FIELD OF DISCLOSURE

(71) Applicant: W. L Gore & Associates, Inc., Newark, DE (US)

(72) Inventor: James L. Goepfrich, Flagstaff, AZ (US)

(73) Assignee: W. L. Gore & Associates, Inc., Newark, DE (US)

(21) Appl. No.: 14/103,319

(22) Filed: Dec. 11, 2013

Related U.S. Application Data
(60) Provisional application No. 61/739,578, filed on Dec. 19, 2012.

(51) Int. Cl. A61M 29/02

(52) U.S. Cl. CPC A61M 29/02
USPC 606/192; 29/456

(57) ABSTRACT

The present disclosure provides devices and methods directed towards threadably coupling a balloon and a tube member to assemble a catheter assembly. For example, in accordance with an embodiment of the present disclosure, an improved catheter assembly comprises a tube member, having a first end and a second end with a lumen extending therebetween, and a balloon, wherein the balloon is in fluid communication with the lumen and is threadably coupled to the tube member.

### FIELD OF DISCLOSURE

The present disclosure relates generally to catheter assemblies having a balloon threadably coupled to the catheter body, and to methods for assembling the same.

### BACKGROUND OF THE DISCLOSURE

Balloon catheters are employed in a wide variety of medical procedures, including dilation of narrowed blood vessels, placement of stents and other implantable devices, temporary or permanent occlusion of blood vessels, drug delivery, thrombectomy, embolectomy, atherectomy, angioplasty, other endovascular procedures, and other procedures within the lumen of a mammalian body such as a human body. In a typical application, a balloon catheter can be advanced to the desired location in the vascular system or other lumen in the body. The balloon is then pressure-expanded in accordance with a medical procedure. Thereafter, the pressure is released from the balloon, allowing the balloon to contract and permitting removal of the balloon catheter.

Balloon catheters are typically formed by coupling an inflatable balloon to a catheter body. The catheter body is typically made of a non-compliant material. In contrast, the inflatable balloon can be made of various materials, depending on whether it will be compliant or non-compliant. The inflatable balloon section can be coupled to the catheter body using adhesives, laser welding, thermal bonding, crimps, shrink tubes, olives, or various other means.

However, commonly used means for coupling the balloon to the catheter body can require increased manufacturing cost and complexity, or can limit the materials used. For example, due to chemical differences, adhesives do not always form strong bonds between compliant materials, such as silicone, and typical catheter-body materials, such as polyurethane. When a compliant balloon is inflated repeatedly, or to high pressures, the balloon can peel away from the catheter body, causing the bond to fail.

Thus, technology that simplifies the process of coupling a balloon to a catheter body is desirable because it can potentially increase production efficiency and balloon catheter quality. Additionally, technology that improves the means of coupling a balloon to a catheter body is desirable because it can potentially improve balloon catheter performance and increase the available materials for manufacture.

### SUMMARY OF THE DISCLOSURE

In general, the present disclosure provides systems and methods for balloon catheter assemblies wherein the balloon is threadably coupled to the catheter body. For example, in various embodiments, a device is provided comprising a tube member having a first end and a second end with a lumen extending therebetween, and a balloon, wherein the balloon is in fluid communication with the lumen and is threadably coupled to the tube member. In further embodiments, methods of assembling or disassembling a balloon catheter are provided.

### BRIEF DESCRIPTION OF THE DRAWINGS

The accompanying drawings are included to provide a further understanding of the present disclosure and are incorporated in, and constitute a part of, this specification, illustrate various embodiments, and together with the description, serve to explain the principles of the disclosure.

FIG. 1a illustrates a perspective view of the disassembled components of a catheter assembly.

FIG. 1b illustrates a perspective view of an assembled catheter assembly.

FIG. 1c illustrates a perspective view of an assembled and inflated catheter assembly.

FIG. 1d illustrates a cross-sectional view of an assembled and inflated catheter assembly.

FIG. 2a illustrates a side view of a catheter assembly, wherein the balloon is viewed cross-sectionally.

FIG. 2b illustrates a side view of a catheter assembly, wherein the balloon is viewed cross-sectionally.

FIG. 3 illustrates a side view of a catheter assembly, wherein the balloon is viewed cross-sectionally.

FIG. 4 illustrates a side view of a catheter assembly, wherein the balloon is viewed cross-sectionally.

FIG. 5 illustrates a side view of a catheter assembly, wherein the balloon is viewed cross-sectionally.

### DETAILED DESCRIPTION

Persons skilled in the art will readily appreciate that various aspects of the present disclosure may be realized by any number of methods and apparatuses configured to perform the intended functions. Stated differently, other methods and apparatuses may be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

As used herein, the term “proximal,” refers to a portion of the catheter assembly closer to the entity assembling, operating, or otherwise using the catheter assembly than a second reference point; the term “distal,” as used herein, refers to a portion of the catheter assembly farther from the practitioner assembling, operating, or otherwise using the catheter assembly than a second reference point. With further regard to the terms proximal and distal, and because the present disclosure is not limited to peripheral and/or central approaches, this disclosure should not be narrowly construed with respect to these terms. Rather, the assemblies described herein can be altered and/or adjusted relative to the anatomy of a patient.

Additionally, catheter bodies used in medical procedures are generally tubes or otherwise cylindrical structures. However, although the terms “catheter body” and “tube member” are used interchangeably herein, other non-cylindrical shapes which incorporate the present disclosure's elements may likewise be used.

Additionally, as used herein, balloon catheters generally refer to medical devices wherein a tube can be inserted into a body cavity, duct, or vessel, and an attached balloon can be inflated hydraulically or pneumatically to, alone or in conjunction with one or more endoluminal devices, enlarge an opening or passage in the body. Accordingly, as used herein, the term “catheter assembly” refers to a device that can be inserted into a body and inflated to enlarge an opening or passage in that body. Briefly, other passage-enlarging devices may likewise benefit from the present disclosure, and use of the term “catheter assembly” herein is not intended to limit the scope, applicability, or configuration of the disclosure in any way.

As used herein, “threads” refer to generally helical or spiral ridges and valleys on one component that are configured to engage generally similar ridges and valleys on another component in order to facilitate securing the components to one another. As used herein, “threads” can be either “right-handed” or “left-handed.”

Numerous materials can be used to achieve each element of the apparatus disclosed herein. Typical materials used to construct the present disclosure can include commonly known materials such as Amorphous Commodity Thermoplastics that include Polymethyl Methacrylate (PMMA or Acrylic), Polystyrene (PS), Acrylonitrile Butadiene Styrene (ABS), Polyvinyl Chloride (PVC), Modified Polyethylene Terephthalate Glycol (PETG), Cellulose Acetate Butyrate (CAB); Semi-Crystalline Commodity Plastics that include Polyethylene (PE), High Density Polyethylene (HDPE), Low Density Polyethylene (LDPE or LLDPE), Polypropylene (PP), Polymethylpentene (PMP); Amorphous Engineering Thermoplastics that include Polycarbonate (PC), Polyphenylene Oxide (PPO), Modified Polyphenylene Oxide (Mod PPO), Polyphenylene Ether (PPE), Modified Polyphenylene Ether (Mod PPE), Thermoplastic Polyurethane (TPU); Semi-Crystalline Engineering Thermoplastics that include Polyamide (PA or Nylon), Polyoxymethylene (POM or Acetal), Polyethylene Terephthalate (PET, Thermoplastic Polyester), Polybutylene Terephthalate (PBT, Thermoplastic Polyester), Ultra High Molecular Weight Polyethylene (UHMW-PE); High Performance Thermoplastics that include Polyimide (PI, Imidized Plastic), Polyamide Imide (PAI, Imidized Plastic), Polybenzimidazole (PBI, Imidized Plastic); Amorphous High Performance Thermoplastics that include Polysulfone (PSU), Polyetherimide (PEI), Polyether Sulfone (PES), Polyaryl Sulfone (PAS); Semi-Crystalline High Performance Thermoplastics that include Polyphenylene Sulfide (PPS), Polyetheretherketone (PEEK); and Semi-Crystalline High Performance Thermoplastics, Fluoropolymers that include Fluorinated Ethylene Propylene (FEP), Ethylene Chlorotrifluroethylene (ECTFE), Ethylene, Ethylene Tetrafluoroethylene (ETFE), Polychlortrifluoroethylene (PCTFE), Polytetrafluoroethylene (PTFE), expanded Polytetrafluoroethylene (ePTFE), Polyvinylidene Fluoride (PVDF), Perfluoroalkoxy (PFA). Other commonly known medical grade materials include elastomeric organosilicon polymers, polyether block amide or thermoplastic copolyether (PEBAX) and metals such as stainless steel and nickel/titanium alloys. Additionally various materials can be combined to obtain the most attractive characteristics of existing (or as yet unknown) plastic, metal, or composite materials, and can be incorporated into the elements disclosed herein.

As noted above, in conventional balloon catheters, the balloon is coupled to the catheter body through various means including adhesives, laser welding, thermal bonding, crimps, olives, and the like. In accordance with the present disclosure, manufacturing efficiencies and quality improvements can be achieved through incorporation of a threaded balloon catheter seal.

For example, a balloon catheter can be improved by coupling the balloon to the catheter body with a threaded seal. Threads located on the catheter or balloon components can increase the pressure-bearing surface area which creates the seal between the catheter body and the balloon. As a result, the seal can be less likely to fail when the balloon is subjected to high pressures during inflation.

Additionally, use of a threaded seal can improve manufacturing efficiencies and ease of assembly. For example, by eliminating the need for additional components such as olives, crimps, and chemical sealants, as well as the need for manufacturing processes such as heat sealing and crimping, cost efficiencies can be achieved. Use of a threaded seal can also ease assembly, allowing users, rather than the manufacturer, to couple the balloon to the catheter and optionally detach the balloon from the catheter, and in some embodiments do so in vivo. As such, balloon catheter components can be manufactured or sold as modular components; and users can assemble different components based on a particular need, while still maintaining the functionality and quality of the balloon catheter seal.

Further, while threadably coupling the balloon to the catheter can create a seal sufficient to withstand high pressure associated with balloon inflation, other methods of creating a balloon catheter seal can be used in conjunction with the present disclosure. For example, a balloon can be threadably coupled to a catheter body and also generally coupled through various means including adhesives, laser welding, thermal bonding, crimps, olives, and the like.

The above being noted, in accordance with an embodiment of the present disclosure, a catheter assembly comprises a tube member having a first end and a second end with a lumen extending therebetween, and a balloon, wherein the balloon is in fluid communication with the lumen and is threadably coupled to the tube member. Briefly, these features of the present disclosure are provided in order that the detailed description herein will be better understood and appreciated; however, the present disclosure can also comprise additional features, which will be subsequently described.

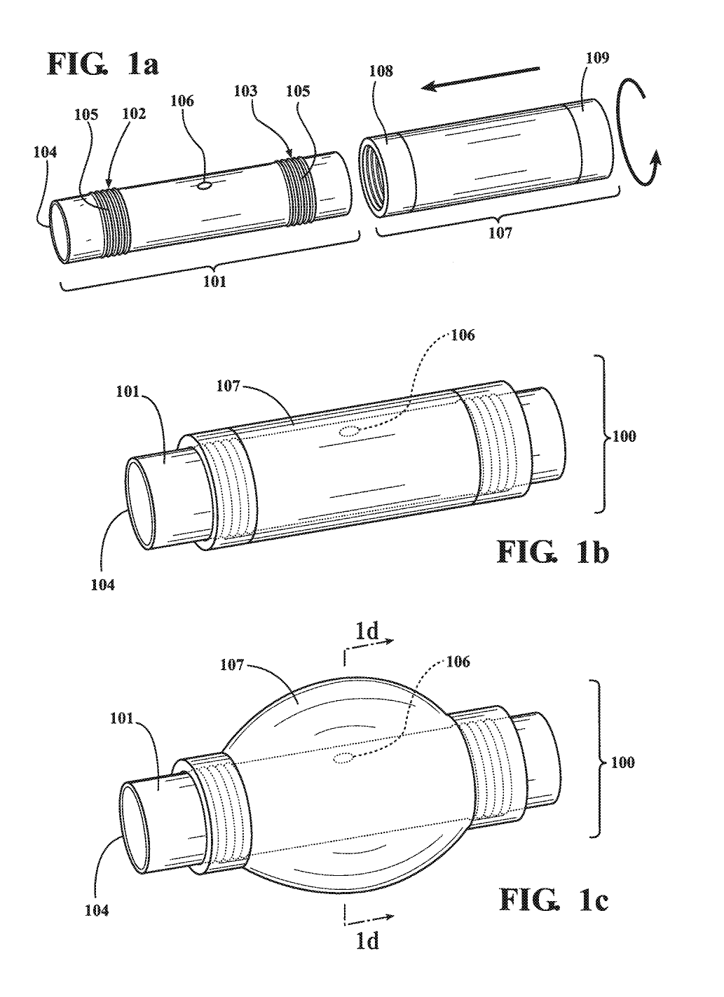

For example, with reference to FIG. 1a, a catheter assembly 100 comprises a tube member 101 and a balloon 107. In an embodiment, the tube member 101 can comprise a first tube end 102 and a second tube end 103, with a lumen 104 extending therebetween. The first tube end 102 generally can be located distally to the second tube end 103. That being said, the tube member ends can be located anywhere on the tube member while still falling within the scope of the present disclosure.

In various embodiments, the tube member 101 can be made with materials such as those previously described herein. However, one skilled in the relevant art will recognize that the tube member can be made of any number of plastics or composite materials. Further, the tube member's physical characteristics, such as flexibility, diameter, wall thickness, and weight can be varied in any way necessary to accomplish the desired performance characteristics, while still falling within the scope of the present disclosure.

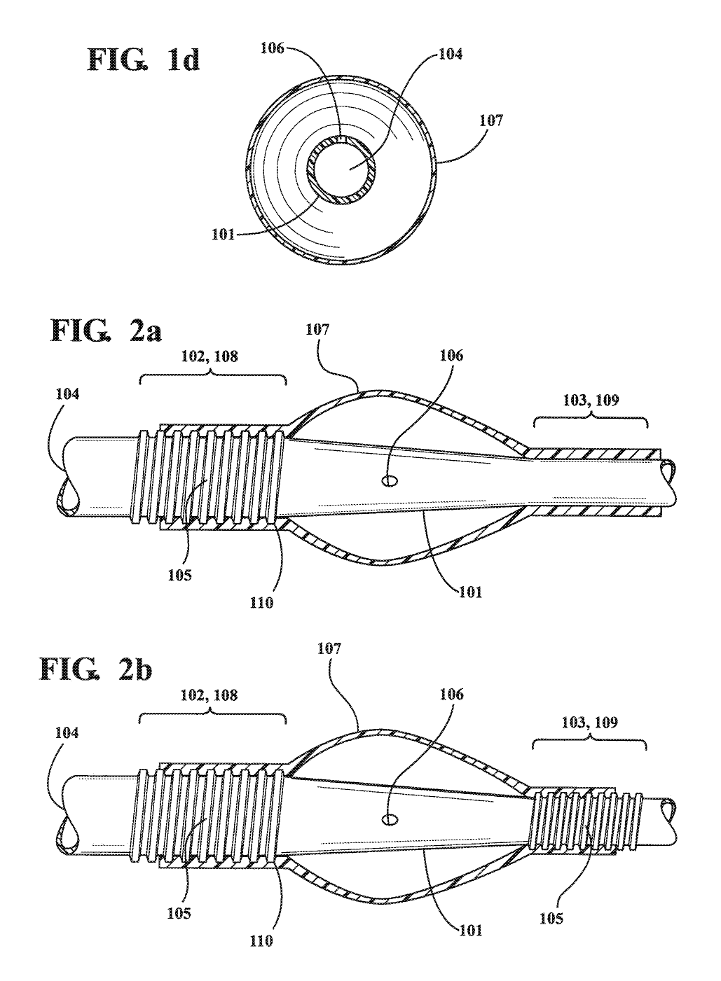

The tube member 101 can be generally cylindrical in shape. However, the tube member can take any number of shapes potentially varying in length, wall-thickness, diameter, or cross-sectional profile. In accordance with the present disclosure, variations in tube member diameter can allow the balloon to slide over portions of the tube member without threadably coupling to it. For example and with reference to FIGS. 2a and 2b, the diameter of the tube member can vary along its length. The tube member diameter can be greater at the first tube end 102 than at the second tube end 103. Alternatively, and with reference to FIG. 4, the tube member diameter can be approximately equal at both tube ends 102, 103, but can narrow between the tube ends 102, 103. That being said, those skilled in the art will recognize that the tube member can take any number of shapes depending on the particular requirements of the catheter assembly, and the embodiments described herein are not intended to limit the scope of the present disclosure.

Additionally, in accordance with the present disclosure and with reference to FIG. 1a, the tube member 101 can comprise tube member threads 105 on at least one of the tube ends 102, 103. The tube member threads 105 can be located on the exterior surface of the tube member 101. Further, the tube member threads 105 can extend axially for a distance along at least one tube end.

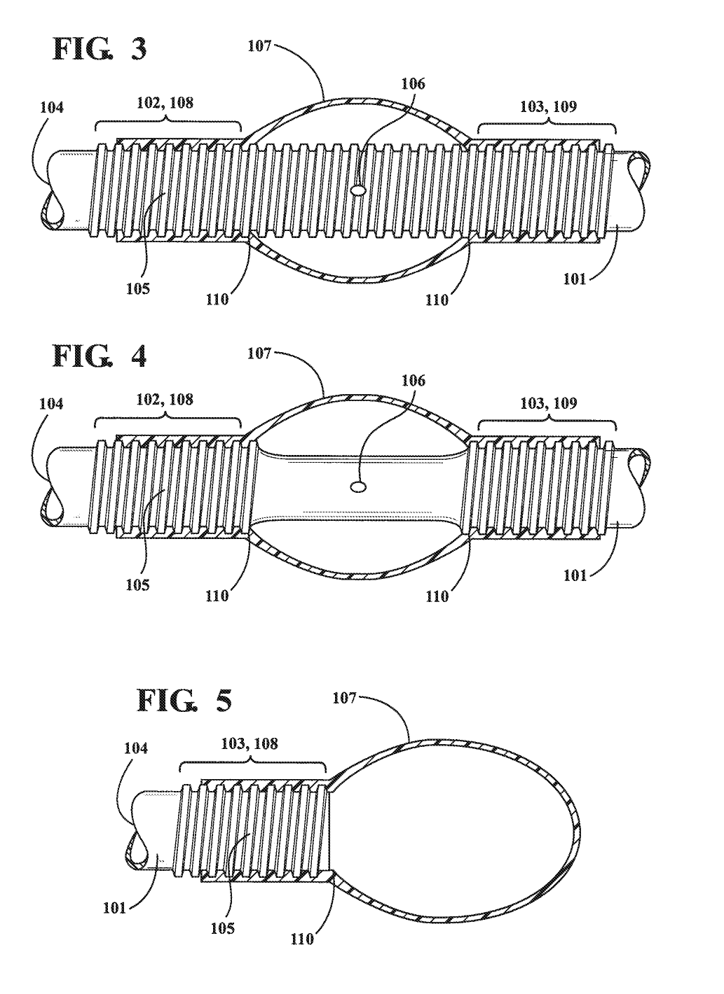

For example, and with reference to FIG. 5, tube member threads 105 can be located only on the second tube end 103. Alternatively and with reference to FIG. 4, tube member threads 105 can be located on both tube ends 102, 103. In yet a further embodiment of the present disclosure, and with reference to FIG. 3, tube member threads 105 can be located on both tube ends 102, 103 and can extend axially, continuously along the tube member 101 between the tube ends 102, 103.

The tube member 101 can additionally comprise an aperture 106. The aperture 106 can be adapted to allow fluid communication (pneumatic or hydraulic) between the lumen 104 and the interior of the balloon 107. Fluid communication can facilitate balloon inflation when the catheter assembly 100 is fully assembled. The aperture 106 generally can be located between the tube ends 102, 103.

That being said, the aperture can be located on any portion of the tube member such that it facilitates balloon inflation. Further, the aperture can vary in size and shape. One skilled in the relevant art will recognize that the aperture can take any number of shapes and sizes depending on the particular requirements of the catheter assembly, and the embodiments described herein are not intended to limit the scope of the present disclosure.

In accordance with an embodiment, the catheter assembly further comprises a balloon 107. In various embodiments, the balloon 107 generally can be made of compliant or non-compliant materials, such as those previous described herein, depending on the particular requirements of the catheter assembly. That being said, catheter balloons can be made of numerous materials. Although an exhaustive list is not included herein, one skilled in the art will appreciate that various plastics, metals, composites, and various combinations thereof can be used, all of which fall within the scope of the present disclosure.

In an assembled catheter assembly, the balloon can at least partially surround the tube member. With reference to FIGS. 1a-1d, the balloon 107 can be adapted to slide over portions of the tube member 101 and to threadably couple with other portions of the tube member 101, such as the tube ends 102, 103 or the tube member threads 105. With reference to FIG. 1b, once the catheter assembly 100 is fully assembled, the balloon 107 can fully surround the tube member 101. Alternatively and with reference to FIG. 5, the balloon 107 can only partially surround the tube member 101 when the catheter assembly 100 is fully assembled.

Additionally and with reference to FIG. 1c, once the balloon 107 is threadably coupled to the tube member 101, the balloon can be inflated by a fluid or a gas. With reference to FIG. 1d, the balloon 107 in its inflated state can be located concentrically around the tube member 101. That being said, the balloon need not be located concentrically around the tube member, but can eccentrically surround the tube member in any number of configurations, all of which fall within the scope of the present disclosure.

The balloon can further comprise at least one balloon end. For example, and with reference to FIG. 5, the balloon 107 can comprise only a first balloon end 108. In such embodiments, the proximal end of the tube member 101 can be open or otherwise comprise an aperture to facilitate inflation of the balloon 107. Alternatively, and with reference to FIG. 1a, the balloon 107 can comprise a first balloon end 108 and a second balloon end 109. The first balloon end 108 generally can be located distally to the second balloon end 109. That being said, the balloon ends can be located on various portions of the balloon, while still falling within the scope of the present disclosure.

In accordance with the present disclosure, the balloon 107 can further comprise balloon threads 110. Balloon threads 110 can be located on the interior surface of the balloon 107. Further, the balloon threads 110 can extend axially for a distance along at least one balloon end. For example, with reference to FIG. 2a, balloon threads 110 can be located on the first balloon end 108 only. Alternatively and with reference to FIG. 2b, balloon threads 110 can be located on both balloon ends 108, 109. Additionally, to facilitate coupling with the tube member, the location and configuration of balloon threads can be complimentary to the tube member threads to which the balloon is coupled. That being said, balloon threads can be located and configured in any manner sufficiently adapted to threadably couple the balloon to the tube member.

In accordance with the present disclosure, the balloon can take any of numerous shapes. In general, the shape of the balloon can be complimentary with the shape of the tube member to which it is coupled and/or an endoluminal device used with the balloon. Complimentary shapes can facilitate creation of a seal between the balloon and the tube member or endoluminal device. For example, with reference to FIGS. 1a-1d, the balloon can be generally cylindrical prior to inflation. That being said, one skilled in the relevant art will appreciate that the shape of the balloon can vary according to the particular needs of the catheter assembly, while still falling within the scope of the present disclosure.

For example, the shape of the balloon, as well as the location, number, and configuration of balloon ends and balloon threads can vary depending on the configuration of the tube member, tube ends, and tube threads. In like manner, the number of balloons can vary. For example, a catheter assembly can comprise a plurality of any combination of the tube member and balloon assemblies described herein.

With reference to FIG. 2a, a catheter assembly can comprise a tube member 101 with a first tube end 102 having a larger diameter than a second tube end 103, and a balloon 107. The tube member 101 can further comprise tube member threads 105 on the first tube end 102. The balloon 107 can be of a size and shape such that the diameter of the first balloon end 108 is larger than that of the second balloon end 109, and is complimentary to the first tube end 102. The balloon 107 can further comprise balloon threads 110 on the inner surface of the first balloon end 108. The balloon 107 and tube member 101 can be thus shaped and configured to allow, during assembly, the first balloon end 108 to pass over the second tube end 103 and threadably couple to the first tube end 102. The second balloon end 109 can be generally coupled to the second tube end 103 through various means including adhesives, laser welding, thermal bonding, crimps, olives, or various other means. With reference to FIG. 2b, the second balloon end 109 can additionally be threadably coupled to the second tube end 103 in alternative embodiments comprising tube member threads 105 or balloon threads 110 on the second tube end 103 and second balloon end 109, respectively.

Alternatively and with reference to FIG. 3, a catheter assembly can comprise a tube member 101 and a balloon 107, with tube member threads 105 extending axially from a second tube end 103 to a first tube end 102. The balloon 107 can comprise balloon threads 110 on both of its balloon ends 108, 109 and be of a size and shape that is complimentary to the tube member 101. The balloon 107 and tube member 101 can be thus shaped and configured to allow, during assembly, the balloon threads 110 located on the first balloon end 108 to threadably couple to the tube member threads 105 located on the second tube end 103 such that rotation of the balloon can result in the balloon surrounding the tube member and achieving fluid communication with the lumen 104 through the aperture 106.

Alternatively and with reference to FIG. 4, a catheter assembly can comprise a tube member 101 and a balloon 107 with balloon threads 110 on both of its balloon ends 108, 109, wherein the balloon's size and shape are complimentary to the tube member 101. The tube member 101 can comprise tube member threads 105 on both tube ends 102, 103. Further, the tube member diameter can be approximately equal at both tube ends 102, 103, but can narrow between the tube ends 102, 103. The balloon 107 and tube member 101 can be thus shaped and configured to allow, during assembly, the first balloon end 108 to threadably couple with the second tube end 103 and, with continued rotation, to decouple from the second tube end 103. Decoupling in this manner can allow the first balloon end 108 to slide distally towards the first tube end 102 and, with rotation, threadably couple to the first balloon end 108 while the second balloon end 109 threadably couples to the second tube end 103.

That being said, the described embodiments are merely illustrations of how the balloon shape and tube member shape can be adapted in various embodiments of the present disclosure. Described embodiments are not intended to limit the scope of the present disclosure in any way. Those skilled in the art will recognize that the embodiments herein are merely examples, and the balloon and tube member can take various shapes and configurations, all while falling within the scope of the present disclosure.

With reference to FIGS. 1a-1c, a method for assembling a catheter assembly 100 is described. The balloon 107 is positioned proximally to the tube member 101 and is moved distally over the second tube end 103 such that the balloon 107 partially surrounds the tube member 101. The balloon 107 is then rotated in the direction of the tube member threads 105 while being moved in a distal direction. This motion is continued until the first balloon end 108 is first adjacent to, and then threadably coupled to, the first tube end 102, while simultaneously the second balloon end 109 is first adjacent to, and then threadably coupled to, the second tube end 103. Rotation can be continued until the threads are sufficiently secured to create a seal between the balloon and the tube member capable of withstanding the pressure required to inflate the balloon. The balloon 107 can then be inflated by hydraulic or pneumatic pressure applied from the catheter lumen 104, through the aperture 106, and into the interior of the balloon 107. Prior to inflation or subsequent to deflation, the rotation of the balloon can be reversed, the balloon can be decoupled from the tube member, and the catheter assembly can be disassembled.

Alternatively and with reference to FIG. 2b, another method for assembling a catheter assembly is described. The balloon 107 is positioned proximally to the tube member 101 and is moved distally over the second tube end 103. During this movement, the balloon threads 110 located on the first balloon end can pass over the tube member threads 105 located on the second tube end 103, without rotating or threadably coupling to them, because the second tube end's diameter is smaller. This motion is continued until the first balloon end 108 is adjacent to the first tube end 102 and the second balloon end 109 is adjacent to the second tube end 103. The balloon 107 is then rotated in the direction of the tube member threads 105 while being moved farther in a distal direction. This rotation is continued until the first balloon end 108 is threadably coupled to the first tube end 102, while simultaneously the second balloon end 109 threadably coupled to the second tube end 103. The balloon 107 can then be inflated by hydraulic or pneumatic pressure applied from the catheter lumen 104, through the aperture 106, and into the interior of the balloon 107.

Alternatively and with reference to FIG. 4, another method for assembling a catheter assembly is described. The balloon 107 is positioned proximally to the tube member 101 and is moved distally over the second tube end 103 until the balloon threads 110 located on the first balloon end 108 are adjacent to the tube member threads 105 located on the second balloon end 109. The balloon 107 is then rotated in the direction of the tube member threads 105 while being moved farther in a distal direction. This rotation is continued until the balloon threads 110 located on the first balloon end 108 are no longer threadably coupled to the tube member threads 105 located on the second balloon end 109. The balloon can then be moved distally without rotation until the first balloon end 108 is adjacent to the first tube end 102 and the second balloon end 109 is adjacent to the second tube end 103. The balloon 107 is then rotated in the direction of the tube member threads 105 while being moved farther in a distal direction. This rotation is continued until the first balloon end 108 is threadably coupled to the first tube end 102, while simultaneously the second balloon end 109 threadably coupled to the second tube end 103. The balloon 107 can then be inflated by hydraulic or pneumatic pressure applied from the catheter lumen 104, through the aperture 106, and into the interior of the balloon 107.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size, and arrangement of parts including combinations within the principles of the invention, to the full extent indicated by the broad, general meaning of the terms in which the appended claims are expressed. To the extent that these various modifications do not depart from the spirit and scope of the appended claims, they are intended to be encompassed therein.

What is claimed is:

1. A catheter assembly comprising:
a tube member having a first tube end and a second tube end with a lumen extending therebetween; and
a balloon,
wherein the balloon is in fluid communication with the lumen,
wherein the balloon comprises a first balloon end, and
wherein the first balloon end is threadably coupled to the tube member.

2. The catheter assembly of claim 1, wherein the balloon surrounds the tube member.

3. The catheter assembly of claim 1, wherein the balloon is located concentric around the tube member.

4. The catheter assembly of claim 2, wherein an outer surface of at least one tube end further comprises threads, and wherein an inner surface of at least one balloon end further comprises threads.

5. The catheter assembly of claim 4, wherein a tube member diameter is greater at the first tube end than at the second tube end, wherein the first balloon end is threadably coupled to the first tube end, and wherein a second balloon end is generally coupled to the second tube end.

6. The catheter assembly of claim 4, wherein the balloon further comprises a second balloon end, and wherein the first and second balloon ends are threadably coupled to the tube member.

7. The catheter assembly of claim 6, wherein a tube member diameter is greater at the first tube end than at the second tube end, and wherein a balloon diameter is greater at the first balloon end than at the second balloon end.

8. The catheter assembly of claim 4, wherein the outer surface of the tube member further comprises continuous threads between the first and second tube ends.

9. The catheter assembly of claim 6, wherein a tube member diameter is smaller between the first and second tube ends.

10. The catheter assembly of claim 1, wherein the tube member further comprises an aperture located between the first and second tube ends.

11. The catheter assembly of claim 1, further comprising a second balloon end generally coupled to the tube member.

12. The catheter assembly of claim 1, wherein the balloon comprises only one balloon end threadably coupled to one tube end.

13. A method of assembling a catheter assembly, comprising:
placing a balloon over a tube member such that the balloon surrounds the tube member;
positioning the balloon such that balloon threads are adjacent to tube member threads;
rotating the balloon until the balloon threads are threadably coupled to the tube member threads.

14. The method of claim 13, further comprising rotating the balloon threads until a seal is created.

15. The method of claim 13, further comprising detaching the balloon from the tube member.

16. A method of assembling a catheter assembly, comprising:
placing a first balloon end over a second tube end, the second tube end having a smaller diameter than a first tube end,
such that balloon threads pass over tube member threads located on the second tube end, and
such that a balloon surrounds a tube member;
positioning the first balloon end adjacent to the first tube end and positioning a second balloon end adjacent to the second tube end,
such that the balloon threads are adjacent to the tube member threads;
rotating the balloon until the balloon threads are threadably coupled to the tube member threads.

17. A method of assembling a catheter assembly, comprising:
sliding a first balloon end over a second tube end, such that balloon threads located on the first balloon end are adjacent to tube member threads located on the second tube end;
rotating a balloon until the balloon threads located on the first balloon end have temporarily, threadably coupled to the tube member threads located on the second tube end;
rotating the balloon farther in the same direction until the balloon threads located on the first balloon end have decoupled from the tube member threads located on the second tube end;
sliding the balloon over a tube member portion having a smaller diameter than the second tube end;
positioning the first balloon end such that the balloon threads located on the first balloon end are adjacent to the tube member threads located on a first tube end, and the balloon threads located on a second balloon end are adjacent to the tube member threads located on the second tube end; and
rotating the balloon until the balloon threads are threadably coupled to the tube member threads.
